(19) United States
(12) Patent Application Publication
(10) Pub. No.: US 2011/0218629 A1
(43) Pub. Date: Sep. 8, 2011

(54) SPINAL STABILIZATION DEVICE

(76) Inventors: Lawrence J. Binder, Miami, FL (US); Stewart Eidelson, Boca Raton, FL (US)

(21) Appl. No.: 12/877,488

(22) Filed: Sep. 8, 2010

Related U.S. Application Data
(60) Provisional application No. 61/310,420, filed on Mar. 4, 2010.

(51) Int. Cl. A61F 2/44

(52) U.S. Cl. 623/17.16

(57) ABSTRACT

A spinal implant device in accordance with the present disclosure is made of a material suitable for human implantation and has a main body having a substantially square periphery. In addition, the spinal implant device has a tapered leading tip on a distal end of the spinal implant device and contiguous with the main body for insertion into a intervertebral space between two vertebral bodies.

### BACKGROUND OF THE INVENTION

Patient's spinal discs collapse for different reasons, including genetics, disease, and trauma. When spinal disc collapse occurs, such collapse can cause pinching of the exiting nerve roots between the discs. Currently, the standard of care is to replace the degenerated disc with an artificial material to restore the distance between the vertebral bodies back to the normal distance, thereby relieving the pressure on the nerve roots.

There are many different approaches that a surgeon can take to access the disc space of the patient, each of which has advantages and disadvantages. For example, during an anterior lumbar interbody fusion, also known as an “ALIF,” the patient is placed supine and the disc area to be replaced is accessed from the abdomen. When operating on some patients it is more desirable to take a posterior approach. One advantage to the posterior approach is that there is generally much less blood loss. However the exiting nerve roots and spinal cord are exposed and at risk damage. During a posterior lumbar interbody fusion, also known as a “PLIF,” the patient is placed prone and the disc area is accessed from either side of the spinal cord. This procedure is at times more difficult in older patients even though it can be less invasive.

### BRIEF DESCRIPTION OF THE DRAWINGS

FIG. 1 depicts an end perspective view of an exemplary spinal implant device in accordance with an embodiment of the present disclosure.

FIG. 2 depicts a perspective view of the other end of the spinal implant device depicted in FIG. 1.

FIG. 3 depicts a proximal end view of the spinal implant device, such as is depicted in FIG. 1.

FIG. 4 depicts a distal end view of the spinal implant device, such as is depicted in

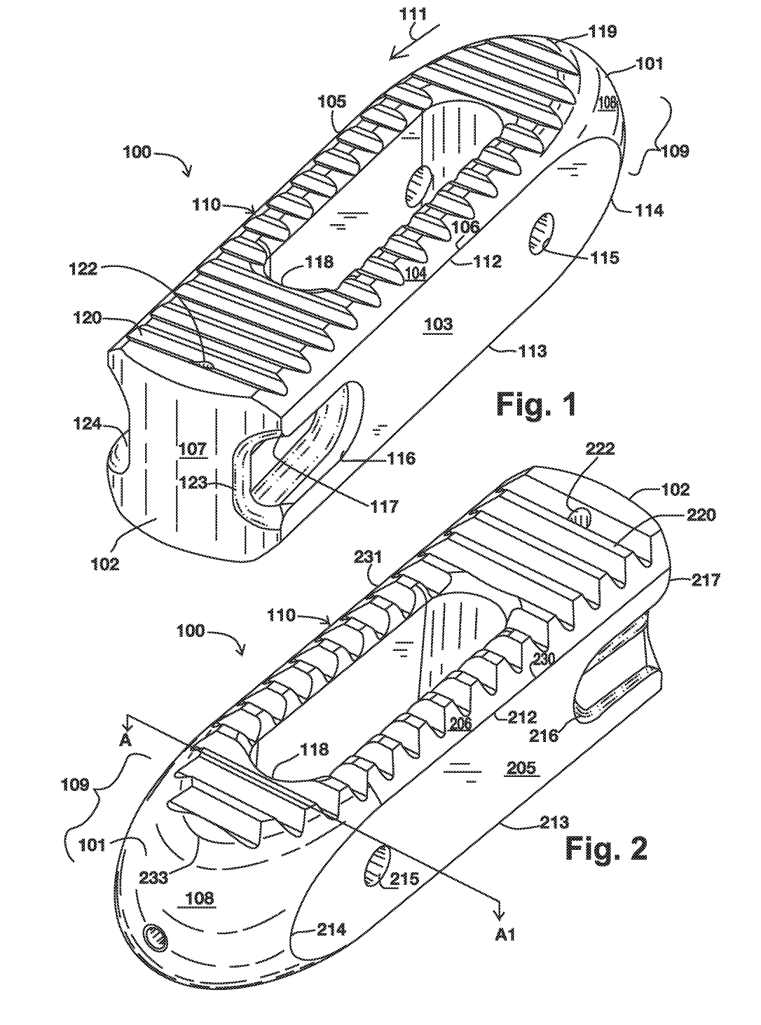

FIG. 1.

FIG. 5A depicts a cross-sectional view of the spinal implant device taken along A-A′ of FIG. 2.

FIG. 5B depicts a cross-section view of another embodiment of a spinal implant device in accordance with the present disclosure.

FIG. 6 depicts an embodiment of an exemplary spinal implant holder tool for holding the spinal implant device depicted in FIG. 1.

FIG. 7 depicts a guide tool inserted between vertebral bodies for receiving a spinal implant device, such as is depicted in FIG. 1.

FIG. 8 depicts the spinal implant holder tool depicted in FIG. 6 inserted into the guide tool depicted in FIG. 7.

FIG. 9 depicts the guide tool after the spinal implant holder tool has been removed from the guide tool and the spinal implant device, such as is depicted in FIG. 1, is fully seated between two vertebral bodies.

FIG. 10 depicts the spinal implant device, such as is depicted in FIG. 1, fully seated between the vertebral bodies with the guide tool removed.

### DETAILED DESCRIPTION OF THE INVENTION

FIG. 1 depicts an end perspective view of an exemplary spinal implant device 100 in accordance with an embodiment of the present disclosure. The spinal implant device 100 comprises a main body 110 that is substantially parallelepiped-shaped with a conical leading tip 109 on a distal end 101. In this regard, the spinal implant device 100 comprises six sides 103, 104, 205 (FIG. 2), 206 (FIG. 2), 107, and 108.

Notably, the spinal implant device 100 has a substantially polygonal cross section. In one embodiment, the substantially polygonal cross section is a square cross section. In another embodiment, the substantially polygonal cross section is a rectangular cross section. In yet another embodiment, the substantially polygonal cross section is a trapezoidal cross section. Other polygonal shaped cross sections are possible in other embodiments of the present disclosure.

The distal end 101, which is the end that is inserted between two vertebral bodies (not shown), is formed by the side 108. The distal end 101 allows for ease of insertion between the vertebral bodies due to the conical leading tip 109. The conical leading tip 109 further functions as a distraction tool while the resulting height of the disc space (not shown) once the device is seated, is greater than before the spinal implant device 100 is implanted. In this regard, the conical leading tip 109 is inserted between the vertebral bodies (not shown), and as the main body 110 is inserted further into the space between the vertebral bodies, the vertebral bodies separate.

The conical leading tip 109 is the conical-shaped portion of the distal end 101, as indicated hereinabove. The conical leading tip 109 inherently has an axial diameter, and the axial diameter of the conical leading tip 109 transitions from smaller to larger as the side 108 approaches the main body 110 of the spinal implant device 100 along reference arrow 111. In this regard, the main body 110 extends outwardly such that it is larger than the axial diameter of the distal-most region of the conical leading tip 109. Such difference in size between the distal-most region of the conical leading tip 109 and the main body 110 ensures that the distance between the vertebral bodies separates as the spinal implant device 100 is inserted between the vertebral bodies.

In one embodiment, the sides 103 and 205 (FIG. 2) are substantially similar. With reference to FIG. 1, the side 103 comprises two parallel edges 112 and 113. The parallel edges 112 and 113 come together at the distal end 101 in the form of an arc-shaped edge 114 that is congruous with the conical tip 109.

In addition, the side 103 comprises a slot 116 formed in an edge 117 of the side 103. In one embodiment the slot 116 extends laterally along the side 103. The slot 116 provides a mechanism by which the spinal fusion device 100 can be inserted between the vertebral bodies. In this regard, the slot 116 receives a tool (not shown) that inserts the spinal fusion device 100 between the vertebral bodies.

Furthermore, the side 103 comprises an opening 115. The opening 115 extends partially through the main body 110 and opens into a hollow opening 118 in the main body 110. The opening 115 is for receiving biological material, such as bone chips, for grafting with the vertebral bodies. In addition, a tool (not shown) for removing the spinal implant device 101 from between the vertebral bodies may be inserted into the opening 115 for ease of removal of the spinal implant device 100.

In one embodiment, the sides 104 and 206 (FIG. 2) are substantially similar. The side 104 comprises two substantially parallel edges 105 and 106 that come together to form an arc-shaped edge 119, which is congruous with the conical leading tip 109. In addition, the side 104 comprises a hollow opening 118 formed therein. The hollow opening 118 can be filled with a biologic material, such as, for example bone chips, for grafting with the vertebral bodies.

Further, the side 104 comprises bone engagement teeth 120 that are slightly slanted toward a proximal end 102 of the spinal implant device 100. The slightly slanted bone engagement teeth 120 allow for ease of insertion in that the bone engagement teeth 120 are slanted away from the distal end 101 and do not interfere with the insertion of the distal end 101 and the body 110 between the two vertebral bodies. However, once the spinal implant device 100 is inserted between the two vertebral bodies, the bone engagement teeth 120 engage the vertebral bodies so as to anchor the spinal implant device 100 between the vertebral bodies by the slanting of the bone engagement teeth 120 toward the proximal end 102. Thus, only by force can the spinal implant device 100 be removed from between the vertebral bodies.

Further, the side 104 comprises an opening 122. The opening 122 extends laterally through the main body 110 and opens on the opposing side 205 (FIG. 2) at opening 222 (FIG. 2). Such opening 222 is for receiving any type of radiopaque marker (not shown), such as, for example a tantalum marker. Thus, the spinal implant device 101 can be located via an x-ray device (not shown) so that positioning of the spinal implant device 100 can be shown on x-ray during a procedure and post-operatively.

The proximal end 102 if formed by the side 107, which is the trailing end of the spinal implant device 100 as the spinal implant device 101 is inserted between the vertebral bodies. In one embodiment, the side 107 has a slightly rounded surface. However, the side 107 may have other types of surfaces in other embodiments, for example the surface may be flat.

The side 107 further comprises indentations 123 and 124, which are extensions of the slots 116 and 216 (FIG. 2), respectively. Such indentations 123 and 124 allow a tool (not shown) access to the slots 116 and 216 for inserting the spinal implant device 100 between the vertebral bodies.

FIG. 2 depicts a perspective view of the end of the spinal implant device 100 opposing that view shown in FIG. 1. FIG. 2 depicts the sides 205, 206, and 108 of the main body 110.

As indicated hereinabove, the distal end 101, which is the end that is inserted between two vertebral bodies (not shown), is formed by the side 108. The distal end 101 allows for ease of insertion between the vertebral bodies due to the conical leading tip 109.

Further, as indicated hereinabove, side 205 is substantially similar to side 103 (FIG. 1). In this respect, side 205 comprises two parallel edges 212 and 213. The parallel edges 212 and 213 come together at the distal end 101 in the form of an arc-shaped edge 214 that is congruous with the conical tip 109.

In addition, the side 205 comprises a slot 216 formed in an edge 217 of the side 205. In one embodiment the slot 216 extends laterally along the side 205. The slot 216 provides a mechanism by which the spinal fusion device 100 can be inserted between the vertebral bodies. In this regard, the slot 216 receives a tool (not shown) that inserts the spinal fusion device 100 between the vertebral bodies.

Furthermore, the side 205 comprises an opening 215. The opening 215 extends partially through the main body 110 and opens into the hollow opening 118 in the main body 110. The opening 215 is for receiving biological material, such as bone chips, for grafting with the vertebral bodies. In addition, a tool (not shown) for removing the spinal implant device 101 from between the vertebral bodies may be inserted into the opening 215 for ease of removal of the spinal implant device 100.

Further, as indicated hereinabove, side 206 is substantially similar to side 104 (FIG. 1). In this respect, the side 206 comprises two substantially parallel edges 230 and 231 that come together to form an arc-shaped edge 233, which is congruous with the conical leading tip 109. In addition, the side 206 comprises the hollow opening 118 formed therein. As described hereinabove, the hollow opening 118 can be filled with a biologic material, such as, for example bone chips, for grafting with the vertebral bodies.

Further, the side 206 comprises bone engagement teeth 220 that are slightly slanted toward the proximal end 102 of the spinal implant device 100. The slightly slanted bone engagement teeth 220 allow for ease of insertion in that the bone engagement teeth 220 are slanted away from the distal end 101 and do not interfere with the insertion of the distal end 101 and the body 110 between the two vertebral bodies. However, once the spinal implant device 100 is inserted between the two vertebral bodies, the bone engagement teeth 220 engage the vertebral bodies so as to anchor the spinal implant device 100 between the vertebral bodies by the slanting of the bone engagement teeth 220 toward the proximal end 102. Thus, only by force can the spinal implant device 100 be removed from between the vertebral bodies.

Further, the side 206 comprises an opening 222. The opening 222 extends laterally through the main body 110 and opens on the opposing side 104 (FIG. 1) at opening 122 (FIG. 1). As described hereinabove, such opening 222 is for receiving any type of radiopaque marker (not shown), such as, for example a tantalum marker.

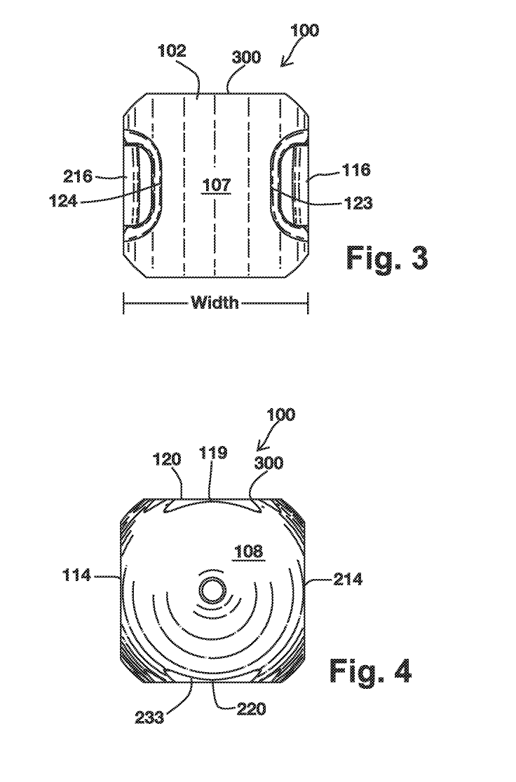

FIG. 3 depicts an end view of the side 107 forming the proximal end 102 of the spinal implant device 100. The end view of the side 107 depicts the indentations 123 and 124 that are formed in the side 107 for receiving an insertion tool for (not shown) for inserting the spinal implant device 100 between the vertebral bodies (not shown).

In addition, the end view of the side 107 depicts the slots 116 and 216 formed in the respective sides 103 (FIG. 1) and 205 (FIG. 2). As described herein, the slots 116 and 216 are also for receiving the insertion tool for inserting the spinal implant device 100 between the vertebral bodies.

FIG. 3 further depicts a substantially square periphery 300. The substantially square periphery 300 is formed by the sides 103 (FIG. 1), 104 (FIG. 1), 205 (FIG. 2), and 206 (FIG. 2) of the main body 110 (FIGS. 1 and 2). The substantially square periphery 300 is such that it fits within an insertion tool (not shown) having a guide tube (not shown) that has a substantially square cross-section.

FIG. 3 further shows the main body 110 having a width that extends from the first side 216 to the second side 116. In one embodiment, the width is in a range from 6 millimeters (mm) to 9 mm.

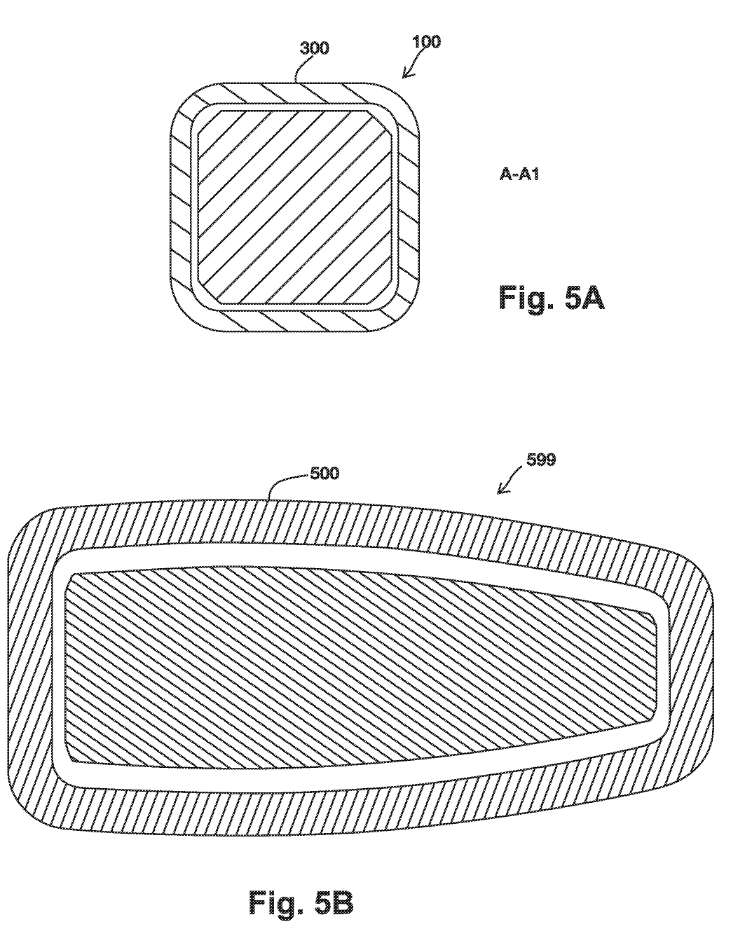

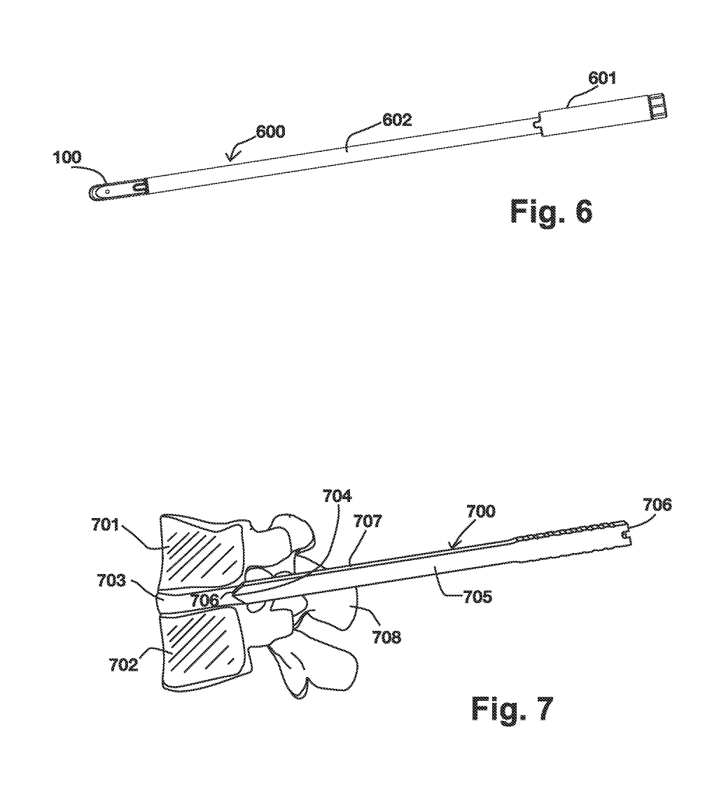

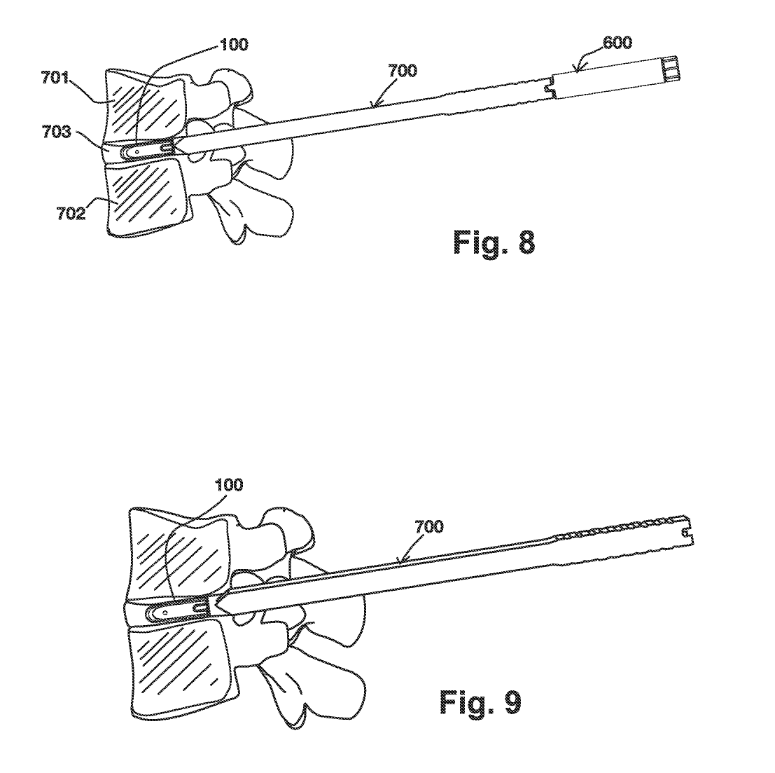

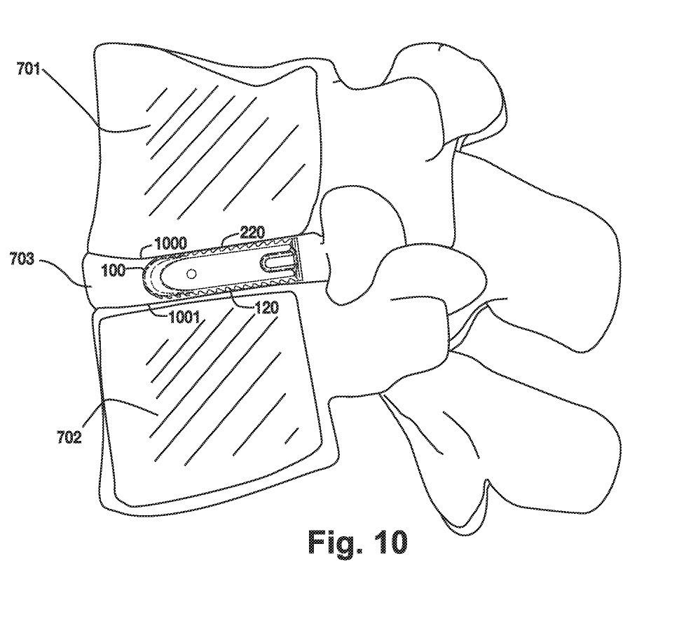

FIG. 4 depicts an end view of the side 108 forming the distal end 101 of the spinal implant device 100. The end view of the side 108 depicts the arc-shaped edges 114, 119, 214, and 233. The edges 114, 119, 214, and 233 are formed by the sides 103 (FIG. 1), 104 (FIG. 1), 205 (FIG. 2), and 206 (FIG. 2), respectively. Further, FIG. 4 depicts the bone engagement teeth 120 and 220. Such teeth 120 and 220 are formed on the sides 104 and 206, respectively.

FIG. 4 further depicts the substantially square periphery 300 of the spinal implant device 100. The substantially square periphery 300 is formed by the sides 103, 104, 205, and 206 of the main body 110 (FIGS. 1 and 2). The substantially square periphery 300 is such that it fits within an insertion tool (not shown) having a guide tube (not shown) that has a substantially square cross-section.

FIG. 5A depicts a cross-sectional view of the spinal implant device 100 taken along A-A′ in FIG. 2. Notably, the cross-sectional view of the spinal implant device 100 depicts the substantially square periphery 300 of the spinal implant device 100.

FIG. 5B depicts a cross-sectional view of a spinal implant device 599 of another embodiment of the present disclosure. In such an embodiment, the spinal implant device 599 has a polygonal shape that is trapezoidal. In this regard, the outer periphery of the spinal implant device 599 is a trapezoidal periphery 500.

FIG. 6 depicts an exemplary implant holder tool 600 in accordance with an embodiment of the present disclosure. The implant holder tool 600 is connected at its distal end to the spinal implant device 100. In one embodiment, the distal end of the implant holder tool 600 is threadedly coupled to the spinal implant device 100. However, other types of coupling devices may be used in other embodiments of the implant holder tool 600 and the spinal implant device 100.

Further, the implant holder tool 600 comprises a handle 601 that is coupled to the distal end via a shaft 602. In one embodiment, the shaft 602 has a square cross-sectional profile for inserting the spinal implant device 100 depicted in FIG. 5A. In another embodiment, the shaft 602 may have a trapezoidal cross-sectional profile for inserting a spinal implant device 599 depicted in FIG. 5B. Further, in one embodiment, the handle 601 may be rotatable such that when the handle 601 is rotated, the implant holder tool 600 threadedly decouples from the spinal implant device 100.

FIG. 7 shows an inferior vertebral body 702 and a superior vertebral body 701 that are separated by the intervertebral space 703. In a natural vertebral body the disc material (not shown) along with other anatomical structures such as the spinous process 708, supports the superior vertebrae. For many reasons, in some people, over time this material degenerates and the distance between the vertebral bodies 701 and 702 lessens collapsing on exiting nerve roots and other surrounding neural elements. This can cause the distance at the posterior portion of the vertebral body bodies 701 and 702 to collapse so much that there is no longer a gap 703 and the vertebral bodies 701 and 702 make contact with each other. Thus, inserting an implant between the vertebral bodies 701 and 702 in the space 703 may be desirable in order to ensure that the vertebral bodies 701 and 702 no longer may direct contact.

Further, FIG. 7 depicts a guide tube 700 that is inserted between the two vertebral bodies 701 and 702 for receiving the sizing tool 600 (FIG. 6). The guide tube 700 is inserted between the two collapsed vertebral bodies 701 and 702 in the intervertebral space 703 between the vertebral bodies 701 and 702. In this regard, guide tube 700 comprises a shaft 705 having a surface 707 and a tip 704 on its distal end. The shaft 705 has a polygonal cross-sectional profile. Thus, the spinal implant device 100 (FIG. 1) can fit snugly through the shaft 705.

Notably, the guide tube 700 has a substantially polygonal cross section. In one embodiment, the substantially polygonal cross section is a square cross section. In another embodiment, the substantially polygonal cross section is a rectangular cross section. In yet another embodiment, the substantially polygonal cross section is a trapezoidal cross section. Other polygonal shaped cross sections are possible in other embodiments of the present disclosure.

The tip 704 of the guide tube 700 is docked between the collapsed vertebral bodies 701 and 702. In this regard, the tip 704 engages the vertebral bodies. In one embodiment, the shape of the tip 704 is such that it fits between the vertebral bodies 701 and 702, locating the center of an intervertebral space 703. In one embodiment, the tip 704 is substantially triangular. In another embodiment, spikes 706 on the tip 704 poke into the surface of the bone on one or both vertebral bodies 701 and 702. In one embodiment, the tip 704 engages a single vertebral body. In another embodiment, the tip 704 engages two vertebral bodies. Notably, the guide tube 700 creates a solid docking location for repeatable measuring and correction of the space 704.

The outer surface 707 of the guide tube 700 shields any surrounding soft tissue and/or neural elements while the spinal implant device 100 is inserted through the inside tubular shaft 705 of the guide tube 700.

FIG. 8 shows the implant holder tool 600 inserting into the shaft 705 of the guide tube 700. In this regard, the implant holder tool 600 is inserted all the way through the shaft 705 toward the posterior region of the collapsed vertebral bodies 701 and 702 so that the spinal implant device 100 rests in the space 703 between the vertebral bodies 701 and 702.

As shown in FIG. 8, the guide tube 700 enters the intervertebral space 703 and distracts the space, thereby reducing pressure on neural elements. Once the spinal implant device 100 is within the intervertebral space 703, the spinal implant device 100 is decoupled from the implant holder tool 600, and the implant holder tool 600 is removed from the shaft, as depicted in FIG. 9.

The guide tube 700 is then removed from the intervertebral space 703 thereby leaving the spinal implant device 100 seated in the intervertebral space 703, as depicted in FIG. 10.

As shown in FIG. 10, endplates 1000 and 1001 of the vertebral bodies 701 and 702 are supported and the spinal implant device 100 has also opened up the posterior region of the space 703. The vertebral bodies 701 and 702 are now supported by the spinal implant device 100 and the surrounding neural elements have been protected and are relieved from impingement. The proximal end of the spinal implant device 100 is positioned recessed from the posterior region of the vertebral bodies 701 and 702 and is generally centered in the intervertebral space 703. The proximal surface of the spinal implant device 100 and the bone engaging teeth 120 and 220 are biased so that they inhibit movement in the opposite axial direction of insertion.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A spinal implant device made of a material suitable for human implantation, comprising:
a main body having a substantially polygonal cross section, wherein the main body comprises a top surface, a bottom surface, and opposing side surfaces;
a tapered leading tip on a distal end of the spinal implant device and contiguous with the main body for insertion into a intervertebral space between two vertebral bodies.

2. The spinal implant device of claim 1, wherein the polygonal cross section is a square cross section.

3. The spinal implant device of claim 1, wherein the polygonal cross section is a rectangular cross section.

4. The spinal implant device of claim 1, wherein the polygonal cross section is a trapezoidal cross section.

5. The spinal implant device of claim 1, wherein the main body comprises a proximal end opposite the distal end, wherein the proximal end comprises an attachment device for attaching to an insertion tool.

6. The spinal implant device of claim 2, wherein the attachment device comprises at least two laterally placed slots located on the opposing side surfaces of the main body.

7. The spinal implant device of claim 3, wherein the attachment device further comprises a threaded hole.

8. The spinal implant device of claim 1, wherein each of the surfaces comprises a plurality of bone engaging teeth.

9. The spinal implant device of claim 8, wherein the plurality of bone engagement teeth slant away from the distal end of the spinal implant device.

10. The spinal implant device of claim 1, wherein the main body comprises an opening extending the top surface of the main body to the bottom surface of the main body.

11. The spinal implant device of claim 10, wherein the opening is adapted for receiving a biological material.

12. The spinal implant device of claim 1, further comprising a guide tube for receiving the main body, the guide tube having inner walls substantially forming a substantially matching polygon.

13. The spinal implant device of claim 12, wherein the inner walls substantially form a square for receiving a main body having a substantially square cross section.

14. The spinal implant device of claim 12, wherein the inner walls substantially form a rectangle for receiving a main body having a substantially rectangular cross section.

15. The spinal implant device of claim 12, wherein the inner walls substantially form a trapezoid for receiving a main body having a substantially trapezoidal cross section.

16. A spinal instrument made of a material suitable for surgical use, comprising:
a substantially parallelepiped tubular body comprising a proximal end, a distal end, and four internal sides that are substantially polygonal in cross section for receiving a spinal implant main body that is substantially polygonal in cross section.

17. The spinal instrument of claim 16, wherein the four internal sides are substantially square in cross section and the spinal implant main body is substantially square in cross section.

18. The spinal instrument of claim 16, wherein the four internal sides are substantially rectangular in cross section and the spinal implant main body is substantially rectangular in cross section.

19. The spinal instrument of claim 16, wherein the four internal sides are substantially trapezoidal in cross section and the spinal implant main body is substantially trapezoidal in cross section.

20. The spinal instrument of claim 16, wherein the distal end has one or more bone engaging means that doc between two vertebrae and the bone engagement means is less than the height of a spinal implant main body to be placed within the parallelepiped tubular body.

21. The spinal instrument of claim 16, wherein the distal end has one or more bone engaging means that engage bony anatomy.

22. The spinal instrument of claim 21, wherein the bone engaging means comprises one or more bone engaging teeth.

23. The spinal instrument of claim 21, wherein the bone engaging means is substantially triangular.

24. The spinal instrument of claim 21, wherein the bone engaging means engages a single vertebrae.

25. The spinal instrument of claim 21, wherein the bone engaging means engages two adjacent vertebrae simultaneously.
